(19) United States
(12) Patent Application Publication
(10) Pub. No.: US 2006/0067993 A1
(43) Pub. Date: Mar. 30, 2006

(54) Topical anesthetic patch

(76) Inventor: Vincent Margiotta, Englewood Cliffs, NJ (US)

Correspondence Address: Donald R. Heiner, Esq., 71 Mount Vernon Street, Ridgefield Park, NJ, 07660, US

(21) Appl. No.: 10/947,715

(22) Filed: Sep. 24, 2004

(51) Int. Cl. A61K 9/70; A61F 13/00

(52) U.S. Cl. 424/449

(57) ABSTRACT

A pre-vaccination or pre-procedural topical anesthetic patch which functions not unlike a nicotine patch and which is essentially a round Band-Aid® containing a topical anesthetic. The Band-Aid® anesthetic carrying device may be of any shape or size. It is applied to a persons skin at a site where a needle is to be inserted such that the site will have a topical anesthetic applied. When the Band-Aid® is removed from the site prior to a needle being inserted a visible delineation of a target area is visible on the skin. That is, that side of the band-aid which contacts the skin will contain a dye, of any color, although preferably red, in the form of a circle or any other geometric shape, smaller than the diameter or size of the band-aid, whereby when the Band-Aid® is removed the dye will leave a mark at the site which is the “target” area. Particularly important is the fact that once the Band-Aid® or patch is removed, prior to a needle being inserted, the target area or bull's eye or a red circle or any other color circle or geometric shape will be left on the skin which clearly delineates where the anesthetic is and, therefore, where the needle should be inserted. Three (3) embodiments are disclosed and each has the same basic elements and can be produced in several different shapes with round or circular being the preferred shape and in addition, the structure can be produced in a square, rectangle, triangle or any other suitable shape.

### BACKGROUND OF THE INVENTION

1. Field of the Invention

Applicant is the owner of U.S. Pat. No. 6,274,167 B1 having a filing date of Sep. 14, 2000 and an application Ser. No. 09/662,169 which issued Aug. 14, 2001 for substantially similar subject matter.

The current invention eliminates item 11 of the U.S. Pat. No. 6,274,167 B1 patent and three (3) embodiments of the invention are disclosed and each has the same basic elements and can be produced in several different shapes with round or circular being the preferred shape. In addition, the device can be produced in a square, rectangle, triangle, or any other suitable shape which will be shown in the drawings and disclosed in the specification.

The present invention generally involves the field of technology pertaining to an anesthetic patch, principally used for babies and small children, but which may be used by others, wherein the device is essentially a common everyday sold over-the-counter preferably round Band-Aid® (trademark) containing a topical anesthetic. The device is placed on a persons skin at a site where a needle is to be inserted such that the area will have a topical anesthetic applied or anesthetic effect. Obviously, the device is applied to the skin in advance of the needle insertion. It should be emphasized that both the Band-Aid® or any other type of bandage material as well as the anesthetic agents are well known and presently approved for over-the-counter sale.

It should be obvious that the anesthetic carrying material may be something other than a Band-Aid® or bandage but can be of other material that will not only carry the anesthetic but will also adhere to a persons skin.

For purposes of further discussing this device the Band-Aid® or bandage or skin contacting anesthetic carrying material will be referred to as an adhesive patch. It is this adhesive patch that will carry any type of topical anesthetic that will prove satisfactory for this purpose and will also carry a dye material on that side of the adhesive patch that contacts the skin. When the adhesive patch is removed, prior to a needle being inserted, it will therefore leave a visible delineation of a “target” area which may be of any geometric shape, although preferably circular, and of any color, preferably red, which clearly marks the spot where a needle etc. is to be inserted.

There are three (3) basic embodiments of this particular disclosure. In the first or preferred embodiment the dye material is disposed on a circular ring of absorbent material which in turn is placed on that side of an adhesive patch that contacts a persons skin. A topical anesthetic is then disposed on the backing and inside the ring.

In a second embodiment the ring and absorbent material are eliminated and the dye is disposed directly on the backing as is the topical anesthetic.

As a third possible embodiment the topical anesthetic may be placed on a solid disk of absorbent material and centrally located such that it may be surrounded by the dye.

This embodiment is the reverse of the preferred embodiment.

It is anticipated that the active ingredient indicated above will be benzocaine and the inactive ingredient indicated above will be glycerin or water or any other benign vehicle. This can be in varying proportions depending upon the age of the recipient.

Any well known dye such as red food dye may be used on the skin side of the adhesive patch to mark the spot to be penetrated. It should be obvious that the active and inactive ingredients can be other than benzocaine and glycerin.

2. Description of the Prior Art

The prior art discussed below is the same prior art that was uncovered during a search prior to the preparation of the application which matured into U.S. Pat. No. 6,274,167 B1.

A search of the prior art has uncovered the following patents: U.S. patent to E. Blank, U.S. Pat. No. 1,682,657; Patel, U.S. Pat. No. 4,162,673; Barrett, U.S. Pat. No. 4,243,035; Haber, U.S. Pat. No. 4,799,926; Grinwald, U.S. Pat. No. 5,003,987; Stalcup, et al, U.S. Pat. No. 5,112,297; Atef, U.S. Pat. No. 5,833,649; and Steinhardt, et al, U.S. Pat. No. 5,902,669.

The patent to Blank, U.S. Pat. No. 1,682,657 is of little interest in that in essence it is really not much more than a brush or any other type applicator and does not comprise an adhesive patch having absorbent cotton and dye.

The patent to Patel, U.S. Pat. No. 4,162,673 is simply a method of testing the position of a needle assembly in the epidural space of a patients body.

Of some general interest is the patent to Barrett U.S. Pat. No. 4,243,035 which is an integral hypodermic syringe and antiseptic dispenser. This would be likened to a pencil having lead at one end and an eraser at the other. This invention is clearly not an adhesive patch soaked with anesthesia and dye which delineates a target area.

Not unlike the Barrett patent discussed above is the patent to Haber U.S. Pat. No. 4,799,926 which in fact cites the Barrett patent. These two patents are seen as being quite similar to each other. This patent is probably even closer to the analogy of a pencil having lead at one end, an eraser at the other, especially as seen in FIG. 3. In this patent the swab 10 is located at and moved around the targeted tissue area 20 of the patient so that an adequate amount of medication will be applied from the swab to the target site. It is after that procedure that the needle is exposed for administering an injection at the target site.

The Grinwald patent, U.S. Pat. No. 5,003,987 entitled “METHOD AND APPARATUS FOR ENHANCED DRUG PERMEATION OF SKIN” requires removal of the outermost layers of the skin by “gentle abrasion” and then measuring the change in the electrical resistence or impedance of the skin as the outmost layers of the skin are removed.

Collectively, the patents to Stalcup, et al, U.S. Pat. No. 5,112,297; Atef, U.S. Pat. No. 5,833,649; and, Steinhardt, et al, U.S. Pat. No. 5,902,669 are of general interest only and fail to disclose the invention described herein.

### SUMMARY OF INVENTION

According to the present invention there is provided an adhesive patch, not unlike current nicotine patches, comprising an adhesive bandage backing, preferably of plastic, but possibly of other materials, which serves as the backing for the adhesive patch of this invention. On that side of the adhesive patch which adheres to the skin is an absorbent material ring, a washer shaped ring disposed on top of said absorbent material, said washer shaped ring further comprising a dye material and, an anesthetic solution disposed on said backing inside said washer shaped ring whereby when said adhesive patch is applied to a persons skin and then removed the skin is anesthetized and a target area for reception of a needle is delineated by said dye operatively associated with said washer shaped ring. This is the preferred embodiment of this invention. Three embodiments of the invention will be disclosed. In all three (3) the backing is plastic.

The anesthetic solution is a benzocaine solution containing both active and inactive solutions.

The anesthetic carrying adhesive patch comprises a benzocaine solution in a crystalline form, polyethylene glycol, sterile water and sorbitol preservative.

In a second embodiment the anesthetic carrying adhesive patch comprises a backing, a washer shaped ring comprising a dye material disposed on one side of said backing, and, an anesthetic solution disposed on said backing inside said washer shaped ring whereby when said adhesive patch is applied to a persons skin and then removed the skin is anesthetized and a target area for reception of a needle is delineated by said dye operatively associated with said washer shaped ring.

In a third embodiment of the invention there is a backing, a solid disk of absorbent material disposed on said backing, an anesthetic solution disposed directly on said solid disk of absorbent material, and, a dye material disposed on said backing surrounding said solid disk of absorbent material. In this embodiment as in the other two (2), when the adhesive patch is applied to a persons skin and then removed the skin is anesthetized and a target area for reception of a needle is delineated by the dye operatively associated with the washer shaped ring.

The dye is preferably a food dye or the like and in any of the embodiments it is that circular shaped dye color the inside of which forms the target area for a needle.

For any of the embodiments the entire device is protected with a plastic or foil strip such that the adhesive patch is kept moist at all times. The entire device is therefore packaged as sterile as are other adhesive bandages.

It is therefore an object of the present invention to provide an anesthetic patch principally used for but not limited to babies and small children.

It is another object of the invention to provide an anesthetic patch which is an adhesive patch to be placed on a person's skin at a site where a needle is to be inserted.

It is yet a further object of the invention to provide such a patch wherein the active and inactive ingredients are preferably benzocaine and glycerine respectively.

It is still a further object of the invention to provide such a patch wherein the active and inactive ingredients may be of varying proportions depending upon the age of the recipient.

It is still a further object of the invention to provide such a patch wherein a skin side of the patch further contains any suitable dye material such that the site on the skin to be penetrated marks the spot of needle insertion.

It is still a further object of the invention to provide such a patch which may be of any desired shape including circular, rectangular, square, triangular, etc.

These and further objects, features and advantages of the invention shall become apparent form the following detailed description of a preferred embodiment thereof when taken in conjunction with the drawings wherein like reference characters refer to corresponding parts in the several views.

### BRIEF DESCRIPTION OF THE DRAWINGS

FIG. 1 is a plan view of the patch showing the skin contacting side of a circular adhesive patch.

FIG. 2 is a cross sectional view taken on line 2-2 of FIG. 1.

FIG. 3 is a plan view of the adhesive patch similar to FIG. 1 without the absorbent material ring of FIG. 2 wherein dye is placed directly on the backing along with the topical solution.

FIG. 4 is similar to FIGS. 1 and 3 but is the reverse of the preferred embodiment shown in FIG. 1.

FIGS. 5 and 6 merely show that any of the three (3) embodiments shown in FIGS. 1, 3 and 4 may be square, rectangular, or any other desired shape.

### DETAILED DESCRIPTION OF THE PREFERRED EMBODIMENT

An anesthetic adhesive patch which may be applied to a persons skin by anyone including the person themselves, a parent, pediatrician, etc. at the site where a needle is to be inserted, for any reason, and which contains a light topical anesthetic solution and which contains both active and inactive ingredients for the purpose of applying a light anesthetic to the skin will now be described with reference to the drawings.

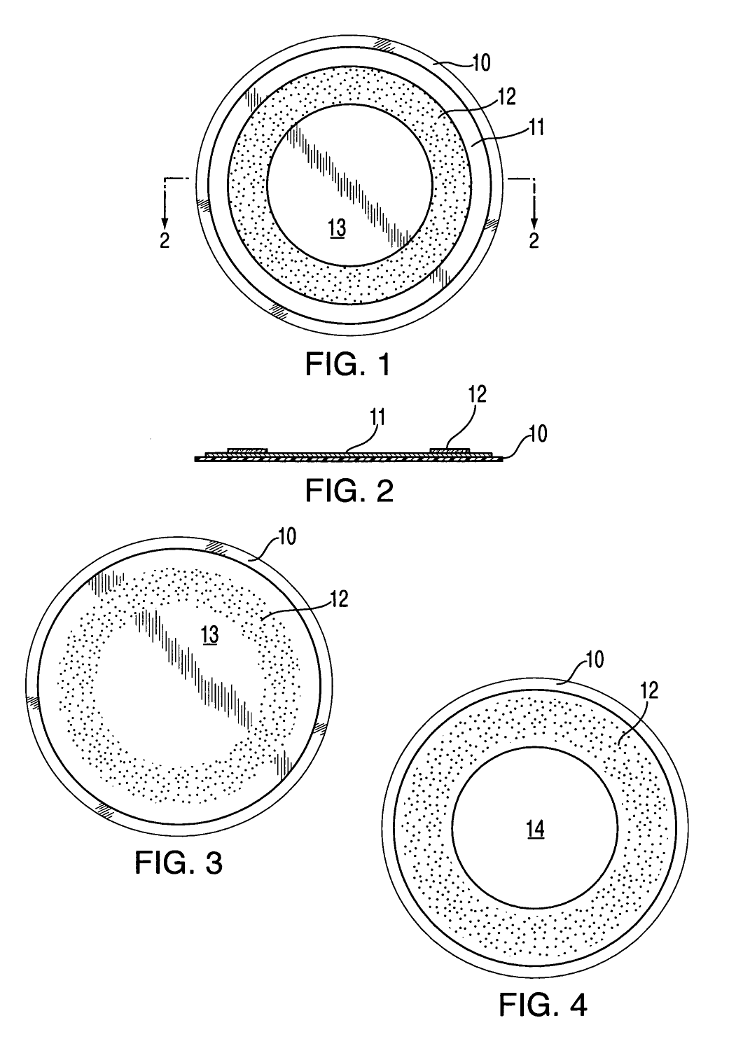

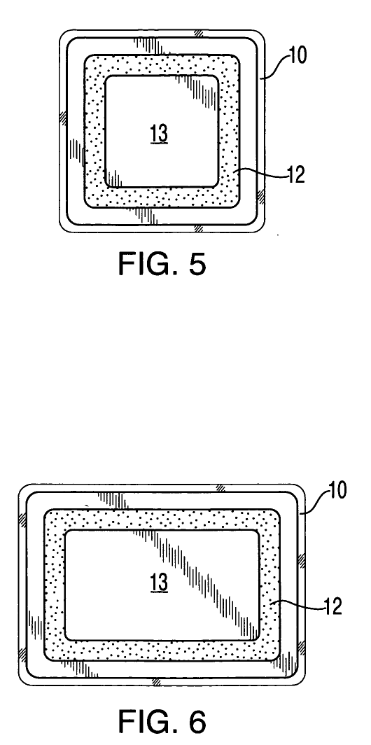

The adhesive patch is shown generally at 10 and in the preferred embodiment shown in FIGS. 1 and 2 there is an absorbent material ring 11 shown disposed on the adhesive patch backing 10 on the side which contacts the persons skin. Disposed on absorbent material circular ring 11 is dye material 12. This dye is of any suitable color, but preferably red, which, in using the adhesive patch 10, as will be more fully described below, will leave a demarcation zone or target area on the skin when the adhesive patch is removed. This target area is where a needle will be inserted.

In this preferred embodiment shown in FIGS. 1 and 2 a topical anesthetic solution 13 of an appropriate percentage of concentration of benzocaine, usually, but not necessarily, in the range of 10%-20%, is applied directly to adhesive patch backing 10 and inside the washer-like ring.

The dye is applied to the backing by any suitable means and may be of any other convenient geometric shape having a non-colored producing center.

The dye is generally and commonly referred to as FD&C #40 (red) although any other colors may be used.

The entire patch described above is preferably covered with a plastic or foil strip to retain moisture and retard evaporation. The entire patch is therefore packaged as sterile as are other over-the-counter adhesive bandages.

The benzocaine solution or topical anesthetic solution 13, preferably of a 10% solution, comprises well known ingredients benzocaine in a crystalline form, polyethylene glycol which acts as a solvent for the crystalline benzocaine, sterile water which is a vehicle for the color, FD&C #40 common food dye and possibly sorbitol preservative.

If the benzocaine solution is to be 10% then 1 gram of crystalline benzocaine (solute) is dissolved into 10 ml of the solvent (polyethylene glycol). If a 20% solution is desired then 2 grams of solute (benzocaine) is dissolved into the same quantity (10 ml) of the polyethylene glycol solvent. Added to the foregoing to yield a final volume of 10 ml is 0.5 ml of the sterile water and 0.5 ml of FD&C #40.

It should be understood that the concentration of the solvent mixture components may be changed, as required, depending upon the desired effect of demarcating the area. Increasing the amount of dye will increase the intensity and duration of the demarcated or target area. However an increase or decrease in any one component will need to be compensated for with a corresponding increase or decrease in one or more of the other components to maintain proper desired stoiciometric percentages.

In addition to the preferred embodiment discussed above there are two (2) other embodiments well within the contemplation of the invention and shown in the drawings and generally discussed above but which will also be discussed further below. In the preferred embodiment the dye is placed on an absorbent ring which can be seen in FIG. 2. The topical solution in turn is placed inside the ring and directly on the backing.

In a second embodiment, the absorbent ring underneath the circular dye is eliminated and the dye is also placed directly on the backing along with the topical solution.

In the third embodiment, a solid disk of absorbent material is placed on the skin contacting side of the adhesive patch and the topical solution is placed directly on this solid disk of absorbent material. The dye is then placed outside this solid disk and surrounding the disk. That is, this is the reverse of the preferred embodiment. The solid disk of absorbent material in this embodiment would still be placed on the backing.

In operation, the adhesive patch is removed from is protective device such as the plastic or foil strip, and is placed on a persons skin, at a predetermined time, and most likely at a predetermined place, with the topical anesthetic solution and the dye material contacting the skin. The adhesive patch backing adheres to the skin. At this time, the skin area under the adhesive patch backing will come into contact with the topical anesthetic agent solution When a needle is to be inserted into the person at the site the patch is removed leaving a demarcated or target area of dye, of any geometric shape, thereby indicating the site for the needle to be inserted. When the patch is removed from the skin it is disposed of.

Though the invention has been described and illustrated with reference to a preferred embodiment thereof, those skilled in the art will appreciate that various changes and modifications in shape, size, composition, and arrangement of parts may be resorted to without departing from the spirit of the invention or scope of the subjoined claims.

What is claimed:

1. An anesthetic carrying adhesive patch attachable to a persons skin comprising:
(a) a backing;
(b) an absorbent material ring disposed on one side of said backing;
(c) a washer shaped ring disposed on top of said absorbent material ring;
(d) said washer shaped ring comprising a dye material; and
(e) an anesthetic solution disposed on said backing inside said washer shaped ring whereby when said adhesive patch is applied to a persons skin and then removed the skin is anesthetized and a target area for reception of a needle is delineated by said dye operatively associated with said washer shaped ring.

2. An anesthetic carrying adhesive patch of claim 1 wherein said backing is plastic.

3. An anesthetic carrying adhesive patch of claim 2 wherein said anesthetic solution is a benzocaine solution.

4. An anesthetic carrying adhesive patch of claim 3 wherein said anesthetic solution contains both active and inactive solutions.

5. An anesthetic carrying adhesive patch of claim 4 wherein said benzocaine solution comprises benzocaine in a crystalline form, polyethylene glycol, sterile water and sorbitol preservative.

6. An anesthetic carrying adhesive patch attachable to a persons skin comprising:
(a) a backing;
(b) a washer shaped ring comprising a dye material disposed on one side of said backing; and
(c) an anesthetic solution disposed on said backing inside said washer shaped ring whereby when said adhesive patch is applied to a persons skin and then removed the skin is anesthetized and a target area for reception of a needle is delineated by said dye operatively associated with said washer shaped ring.

7. An anesthetic carrying adhesive patch of claim 6 wherein said backing is plastic.

8. An anesthetic carrying adhesive patch of claim 7 wherein said anesthetic solution is a benzocaine solution.

9. An anesthetic carrying adhesive patch of claim 8 wherein said anesthetic solution contains both active and inactive solutions.

10. An anesthetic carrying adhesive patch of claim 9 wherein said benzocaine solution comprises benzocaine in a crystalline form, polyethylene glycol, sterile water and sorbitol preservative.

11. An anesthetic carrying adhesive patch attachable to a persons skin comprising:
(a) a backing;
(b) a solid disk of absorbent material disposed on said backing;
(c) an anesthetic solution disposed directly on said solid disk of absorbent material; and
(d) a dye material disposed on said backing surrounding said solid disk of absorbent material whereby when said adhesive patch is applied to a persons skin and then removed the skin is anesthetized and a target area for reception of a needle is delineated by said dye operatively associated with said washer shaped ring.

12. The anesthetic carrying adhesive patch of claim 11 wherein said backing is plastic.

13. The anesthetic carrying adhesive patch of claim 12 wherein said anesthetic solution is a benzocaine solution.

14. The anesthetic carrying adhesive patch of claim 13 wherein said anesthetic solution contains both active and inactive solution.

15. The anesthetic carrying adhesive patch of claim 14 wherein said benzocaine solution comprises benzocaine in a crystalline form, polyethylene glycol, sterile water and sorbitol preservative.

16. An anesthetic carrying adhesive patch attachable to a persons skin comprising:
(a) a backing;
(b) an absorbent material ring disposed on one side of said backing;
(c) a dye material impregnated into said absorbent material ring;
(d) an anesthetic solution disposed on said backing inside said absorbent material ring whereby when said adhesive patch is applied to a persons skin and then removed the skin is anesthetized and a target area for reception of a needle is delineated by said dye operatively associated with and impregnated into said absorbent material ring.
